(19) United States
(12) Patent Application Publication
(10) Pub. No.: US 2016/0106432 A1
(43) Pub. Date: Apr. 21, 2016

(54) SYSTEMS AND METHOD FOR LEFT ATRIAL APPENDAGE OBLITERATION

(71) Applicant: MICROACCESS INC., Weston, MA (US)

(72) Inventor: Sameh Mesallum, Boston, MA (US)

(21) Appl. No.: 14/721,767

(22) Filed: May 26, 2015

Related U.S. Application Data
(60) Provisional application No. 62/005,352, filed on May 30, 2014.
(60) Provisional application No. 62/005,250, filed on May 30, 2014.

(51) Int. Cl. A61B 17/12; A61B 17/00

(52) U.S. Cl. CPC A61B 17/12013; A61B 17/00234; A61B 2017/00243; A61B 2017/00358

(57) ABSTRACT

Various aspects and embodiments solve at least some of the problems associated with conventional LAA closure approaches. Example devices and methods are disclosed for closing the LAA that can be used under direct visual guidance and in a short time period. Various embodiments employ a transesophageal barrier to provide a sealable opening through a patient's esophagus. The sealable opening allows surgical devices and in particular LAA closure devices to pass through the patient's esophagus into the patient's thoracic cavity. In one embodiment, an LAA closure device includes a snare wire having engagement members for maintaining and/or decreasing a circumference of a loop of the snare wire. The loop of the snare wire can be used like a noose to strangle the LAA to achieve closure. In other examples, wire traction devices can be used to close or obliterate the LAA.

### RELATED APPLICATIONS

This application priority under 35 U.S.C. §119(e) to U.S. Provisional Patent Application Ser. No. 62/005,352 entitled “SYSTEMS AND METHOD FOR LEFT ATRIAL APPENDAGE OBLITERATION,” filed May 30, 2014; and to U.S. Provisional Patent Application Ser. No. 62/005,250 entitled “SYSTEMS AND METHOD FOR MICROACCESS TRANSESOPHAGEAL SURGERY,” filed May 30, 2014, which applications are incorporated herein by reference in their entirety.

### BACKGROUND

Recent articles have been published regarding prevention of stroke in patients with atrial fibrillation. Such patients are often unable to use conventional medication to treat or mitigate the risks of stroke. Some approaches have found success in closure of the left atrial appendage (“LAA”) to prevent strokes in these patients. Conventional techniques associated with LAA closure use a device known as the Watchman device. The Watchman device employs multiple hooks to collapse the LAA from within the patient's heart. In particular, the Watchman device is introduced through the patient's groin via a catheter that traverses the inferior vena cava to reach the right atrium (“RA”). Upon reaching the RA a hole is made in the interatrial septum to reach the left atrium (“LA”). The tip of the catheter is inserted into the LAA deploying multiple hooks into the walls of the LAA. Responsive to pulling on the wires connected to the hooks, the LAA cavity is closed or obliterated.

### SUMMARY

It is realized that current LAA treatment approaches and devices are too invasive, too complicated, and the procedure for closing the LAA can be too long, and difficult to complete. Further, complicating the procedure, conventional approaches are done under fluoroscopy guidance making the end to end process difficult to monitor. Accordingly various aspects and embodiments solve at least some of the problems associated with conventional LAA closure approaches. Provided are methods and devices for LAA closure that use a minimally invasive and intra-thoracic approach to the heart. In particular, provided are devices and methods for closing the LAA that can be executed under direct visual guidance and in a short time period. Various embodiments employ a transesophageal barrier that is used to provide a sealable opening through a patient's esophagus. The sealable opening is used to allow surgical devices and in particular LAA closure devices to pass through the patient's esophagus into the patient's thoracic cavity to have access to external portions of the patient's heart. In one embodiment, an LAA closure device includes a snare wire having engagement members for maintaining and/or decreasing a circumference of a loop of the snare wire. The loop of the snare wire is positioned around the LAA until it reaches the connection between the LA and the LAA. Once in place, the snare wire is contracted to compress the LAA at the LA and LAA junction, closing the LAA and/or obliterating the LAA.

According to another embodiment, co-centric rings can be employed as part of an LAA closure device. In one example, the two rings with opposing recurved teeth are connected by circumferential surgical wires along the circumference of both rings. In one example, the LAA closure device can be deployed from a 2-3 mm channel in a scope under visual guidance. A distal ring can be anchored on the neck of the LAA (e.g., at the junction of the LA and LAA) using the recurved teeth. A proximal ring (proximal to the scope) is advanced and anchored into the tissues of the LAA. By retracting the wires connecting the two rings, the LAA is closed and/or obliterated.

According to one aspect an apparatus for executing a left atrial appendage operation is provided. The apparatus comprising a surgical instrument constructed and arranged to pass through a sealable opening at an esophageal barrier, including a closure component configured to attach to an exterior portion of a patient's heart, and to collapse a portion of the heart at the exterior portion of the patient's heart to close the left atrial appendage (“LAA”).

According to one embodiment, the closure component comprises a snare wire configured to extend about the portion of the patient's heart. According to one embodiment, the snare wire comprises a wire having a loop portion, a locking portion, and a plurality of teeth. According to one embodiment, the plurality of teeth are constructed and arranged to directionally engage the locking portion. According to one embodiment, the plurality of teeth are constructed and arranged to pass through the locking potion in one direction and prevent passage through the locking portion in an opposite direction. According to one embodiment, the locking portion is configured to engage subsequent ones of the plurality of teeth to reduce the loop portion in size responsive to retraction of the snare wire. According to one embodiment, the plurality of teeth are constructed and arranged such that the loop portion is reduced to a diameter of less than 2 mm.

According to one embodiment, the closure component comprises at least a first ring and a second ring. According to one embodiment, the at least a first ring and a second ring comprise at least a distal ring and a proximal ring, wherein the distal ring is configured to distend, such that the distal ring is configured to be advanced over tissue of the LAA, such that a portion of the tissue of the LAA pass through the distal ring. According to one embodiment, the distal ring comprises a plurality of unidirectional anchors. According to one embodiment, the plurality of unidirectional anchors are constructed and arranged such that the plurality of unidirectional anchors are oriented to engage the tissue of the LAA and anchor the distal ring against a movement of the distal ring in an opposite direction of the advancement of the distal ring over the tissue of the LAA. According to one embodiment, the proximal ring includes a plurality of anchors configured to engage tissue of the LAA.

According to one embodiment, the apparatus further comprises connection wires between the distal and the proximal rings. According to one embodiment, the connection wires are configured to apply radial pressure to the tissue of the LAA to close the LAA.

According to another aspect, a method for a left atrial appendage operation is provided. The method comprises introducing a surgical device through a sealable opening in an esophageal barrier into a cardiac cavity, advancing a closure component of the surgical device into a thoracic cavity of a patient's heart, capturing, from the thoracic cavity, a portion of a patient's heart including a left atrial appendage with the closure component, and collapsing the portion of the patient's heart using the closure component.

According to one embodiment, capturing, from the thoracic cavity, the portion of a patient's heart with the closure component includes extending a snare wire about the portion of the patient's heart. According to one embodiment, the method further comprises retracting the snare wire, thereby constricting the portion of the heart of the patient. According to one embodiment, capturing, from the thoracic cavity, the portion of a patient's heart with the closure component includes extending a distal ring over the tissue of the LAA. According to one embodiment, the method further comprises anchoring the distal ring at the junction between the LAA and the left atrium (“LA”). According to one embodiment, the method further comprises anchoring a proximal ring on a surface of the LAA. According to one embodiment, collapsing the portion of the patient's heart using the closure component includes exerting radial pressure on the tissue of the LAA with wires connecting the distal and proximal rings.

Still other aspects, embodiments and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Any embodiment disclosed herein may be combined with any other embodiment. References to “an embodiment,” “an example,” “some embodiments,” “some examples,” “an alternate embodiment,” “various embodiments,” “one embodiment,” “at least one embodiment,” “this and other embodiments” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

### BRIEF DESCRIPTION OF THE DRAWINGS

Various aspects of at least one embodiment are discussed below with reference to the accompanying figures, which are not intended to be drawn to scale. Where technical features in the figures, detailed description or any claim are followed by reference signs, the reference signs have been included for the sole purpose of increasing the intelligibility of the figures, detailed description, and claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. The figures are provided for the purposes of illustration and explanation and are not intended as a definition of the limits of the invention. In the figures:

FIG. 1 is an example LAA closure device, according to one embodiment;

FIGS. 2A-B show portions of example LAA closure devices, according to one embodiment;

FIG. 3 illustrates an LAA closure procedure according to one embodiment;

FIG. 4 illustrates a catheter for introducing an LAA closure device, according to one embodiment;

FIG. 5 illustrates an example snare wire, according to one embodiment;

FIG. 6 illustrates an example snare wire, according to one embodiment;

FIG. 7 illustrates an example snare wire post LAA closure, according to one embodiment;

FIG. 8 illustrates an example snare wire secured to a patient's heart according to one embodiment;

FIG. 9 is a semitransparent view of a LAA closure device, according to one embodiment;

FIG. 10 is a semitransparent view of a LAA closure device, according to one embodiment;

FIG. 11 illustrates an example closure device according to one embodiment;

FIG. 12 illustrates an example closure device according to one embodiment;

FIG. 13 illustrates an example closure device according to one embodiment;

FIG. 14 illustrates an example of a surgical device and barrier for performing minimally evasive surgery, according to one embodiment;

FIG. 15 is a side cut out view of an example barrier, according to one embodiment;

FIG. 16 is cut out view of an example barrier, according to one embodiment;

FIG. 17 is view of a surgical device extended through a barrier, according to one embodiment;

FIG. 18 is a view of an example barrier, according to one embodiment;

FIG. 19 is an example process flow for minimally invasive surgery through a body lumen, according to one embodiment; and

FIGS. 20A-B illustrate example barriers in open and closed positions, according to some embodiments.

### DETAILED DESCRIPTION

Stated broadly various aspects and embodiments are directed to minimally invasive transesophageal surgical procedures. In various embodiments, an esophageal barrier is provided that manages sterile access through the esophagus and into a patient's thoracic cavity. Using this approach, surgical procedures can be performed on the patient's heart with direct visualization of the entire procedure. According to one embodiment, an LAA closure device can be carried by a catheter into the patient's esophagus through the barrier to deploy the LAA closure device in the patient's thoracic cavity. The LAA closure device can be deployed around the LAA of the patient's heart. The closure device can be positioned at the junction of the LAA and the LA of the heart. Once in position the closure device can be operated to seal the LAA off from the other portions of the heart, closing and/or obliterating the LAA.

According to some embodiments, the LAA closure device can include a snare wire that is configured with a loop portion. The loop portion can include a locking member and a plurality of engagement portions disposed along the length of the snare wire. By advancing the locking member and/or retraction the snare wire once in position around the LAA, the circumference of the loop is reduced until the LAA is sealed off from the patient's heart effectively obliterating the LAA. In other embodiments, the LAA closure device can include co-centric rings that are deployed around the LAA. A first ring can include recurved teeth that anchor the first ring at the junction between the LAA and LA. A second ring can be connected to the first through wires, such that retraction of the wires obliterates the LAA as the first and second rings are forced together.

Regardless of the LAA closure device used, the catheter carrying the closure device can then be withdrawn from the thoracic cavity, and the esophageal barrier can seal the opening between the esophagus and the thoracic cavity. Thus, as described herein, LAA closure can be accomplished as a minimally invasive procedure, and in some examples without need of general anesthesia.

Examples of the methods, devices, and systems discussed herein are not limited in application to the details of construction and the arrangement of components set forth in the following description or illustrated in the accompanying drawings. The methods and systems are capable of implementation in other embodiments and of being practiced or of being carried out in various ways. Examples of specific implementations are provided herein for illustrative purposes only and are not intended to be limiting. In particular, acts, components, elements and features discussed in connection with any one or more examples are not intended to be excluded from a similar role in any other examples.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

Various embodiments enable procedures that can safely and quickly operate to obliterate or close a portion of a patient's heart, specifically the LAA of the patient's heart. Current treatment studies for prevention of stroke have shown that LAA obliteration can be effective and even superior to conventional anticoagulation treatment. However, conventional implementations for such procedures are overly complicated, time consuming, invasive, and involve operating on the LAA from positions internal to the patient's heart. Accordingly, provided are LAA closure devices that can be employed in minimally invasive approaches, and devices that can operate on the LAA external to the patient's heart

Shown in FIG. 1 is an example embodiment of an LAA closure device 100 that can be introduced into a patient's thoracic cavity through a surgical device (e.g., scope). The LAA closure device can include a snare wire 102 that is configured to include a loop portion such that the loop portion can be position around the patient's LAA. In some examples, the surgical device can be advanced into the patient's thoracic cavity and the snare wire 102 can be deployed such that the loop portioned 104 is positioned around the LAA portion of the patient's heart. In some embodiments, the snare wire includes an engagement member 106 configured to attach to a proximal portion 108 of the snare wire to form the loop portion 104. The engagement member 106 is configured to engage and slide along the length of the snare wire 102 to provide the loop portion 104 which can have a varying circumference. A distal portion 110 of the snare wire (distal from the surgical device carrying the snare wire) can include a plurality of locking elements or stoppers (e.g., at 112). The locking elements are configured to allow the engagement member to advance over the engagement element but not to travel back. For example, the locking elements are configured to allow the circumference of the loop portion 104 to shrink but not increase in size (within some small variation). In some embodiments, as the snare wire is withdrawn the engagement member advanced over the engagement element reducing the circumference of the loop portion 104 until the LAA is closed. Such closure prevents normal blood flow to the closed tissue and the LAA tissue eventually dies, healing at the closure.

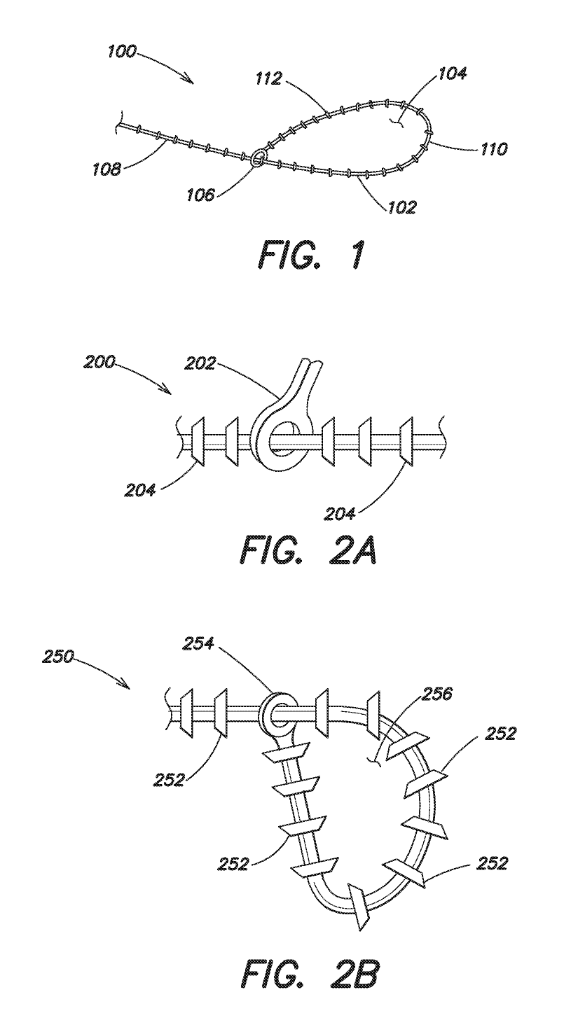

FIGS. 2A-B show a portion of a snare wire 200 and 250 respectively. At 202 an engagement member is shown coupled to the snare wire 200. A plurality of locking members 204 are provided along the length of the snare wire to enable complete closure of the LAA. In some embodiments the spacing of the locking members can vary along the length of the snare wire. For example, at the portions of the snare wire where the circumference of the loop portion approaches a complete closure circumference, the distance between the locking members can be reduced to allow smaller changes in circumference while locking the engagement member in place. Shown in FIG. 2B is another example snare wire 250 and locking members 252. The locking members can be constructed with a stiff interior portion and a flexible outer circumference to enable the engagement member 254 to pass over upon the application of sufficient force. The flexible outer portions can also be configured to resist the reverse motion of the engagement member along the snare wire to prevent the loop portion 256 from increasing in diameter.

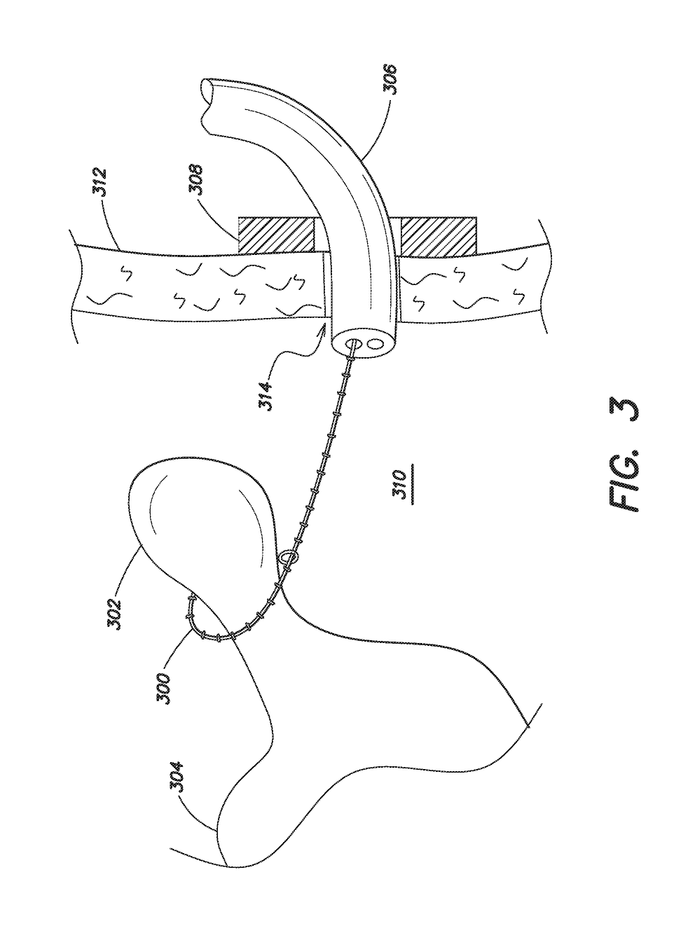

FIG. 3 is a view of an example snare wire 300 deployed around the LAA 302 of a patient's heart at the junction between the LA 304 and the LAA 302. In some embodiments, a surgical device 306 (e.g., a scope) can be advanced into a patient's esophagus until reaching an esophageal barrier 308. Various embodiments and features of an esophageal barrier are described within co-pending U.S. patent application Ser. No. 14/721,492 entitled “SYSTEMS AND METHOD FOR MICROACCESS TRANSESOPHAGEAL SURGERY,” filed May 26, 2015, incorporated by reference in its entirety herein. The esophageal barrier 308 is configured to provide access to the patient's thoracic cavity 310 through an opening 314 in the esophageal wall 312. In some examples, the surgical device can be used to create the opening 314. Once advanced through the opening 314 and into the thoracic cavity 310, the snare wire 300 can be deployed around the LAA of the patient's heart. For example, a loop portion 316 can be positioned around the LAA at the junction between the LAA 302 and the LA 304. According to some embodiments, the surgical device can include multiple channels which house multiple surgical implements (e.g., cutting tools, scalpels, suturing tools, etc.).

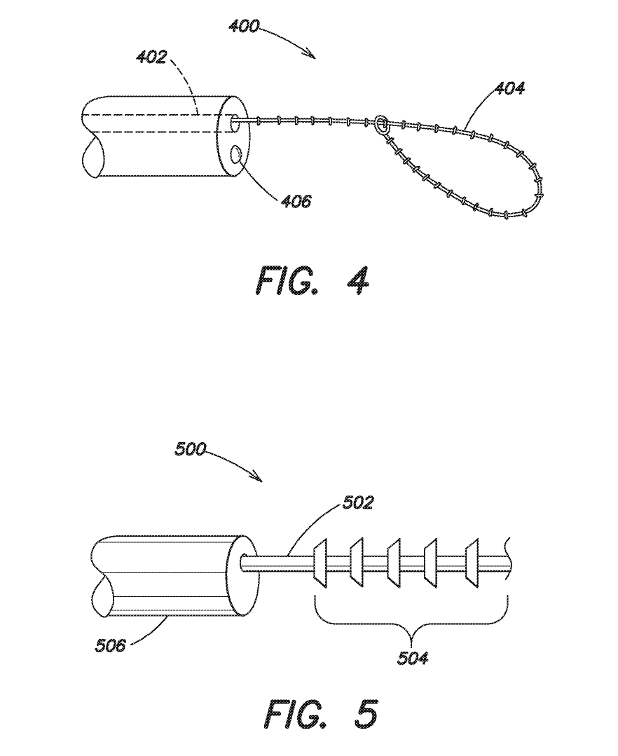

In some embodiments, the surgical device can also include a camera on a distal end of the device. According to one aspect, the surgical device can provide for direct visualization during an LAA closure procedure. FIG. 4 is an illustration of a surgical device 400 having a channel 402 configured to house a LAA closure device 404 and a CCD 406 on a distal end. The CCD can be configured to provide image data in real time to a surgeon and/or operator of the surgical device. Various embodiments of the LAA closure device can include different structures to position the device around the LAA and effect closure. FIG. 5 is a illustration of another embodiment of a LAA closure device 500 including a snare wire 502 with serrated teeth 504 positioned at a distal end of the snare wire (distal to the surgical device 506). The serrated teeth 504 can be configured to provide more secure engagement with an engagement member of, for example, a loop portion of the snare wire.

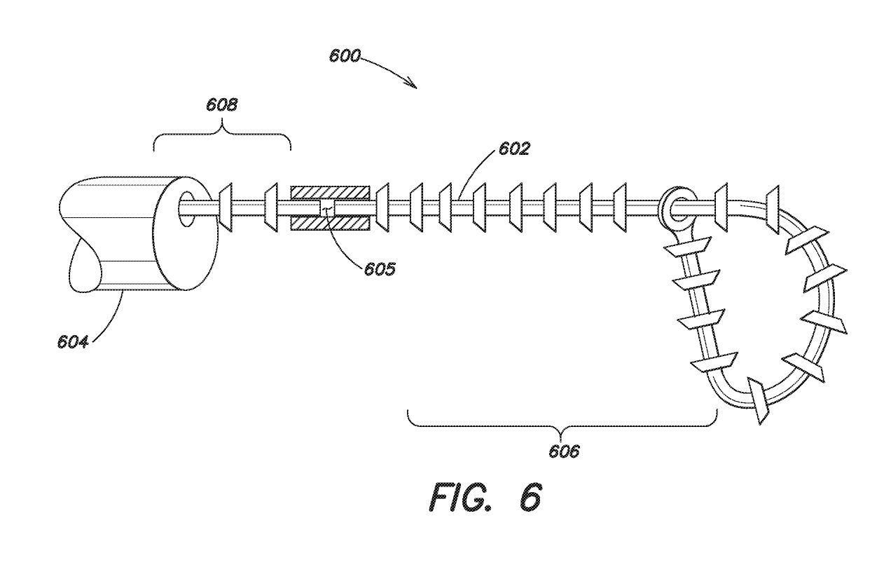

FIG. 6 is another embodiment of a LAA closure device 600. In some embodiments, the snare wire 602 can include a pre-configured break point (e.g., at 605) for separating the snare wire from the surgical device 604. The break point 605 in the snare wire can be used to allow the distal end 606 of the snare wire to be left in place within the patient, while the surgical device 604 and proximal end 608 of the snare wire are withdrawn. In some embodiments, the break point 605 can include a casing configured to hold the ends of the break together. In response to manipulation the casing can be configured to release a connection between the proximal and distal ends of the snare wire. In one example, the casing is connected by a first threaded portion received by a second threaded portion. In response to rotation the first threaded portion and second threaded portion can be released allowing the proximal and distal ends of the snare wire to separate. Other securing mechanisms can be used to keep the break point connected (e.g., pressure fit connections, teeth, lock and key connectors, etc.). In further embodiments, the break point can be a weakened section of the snare wire that is pre-configured to break upon manipulation or application of a cutting device.

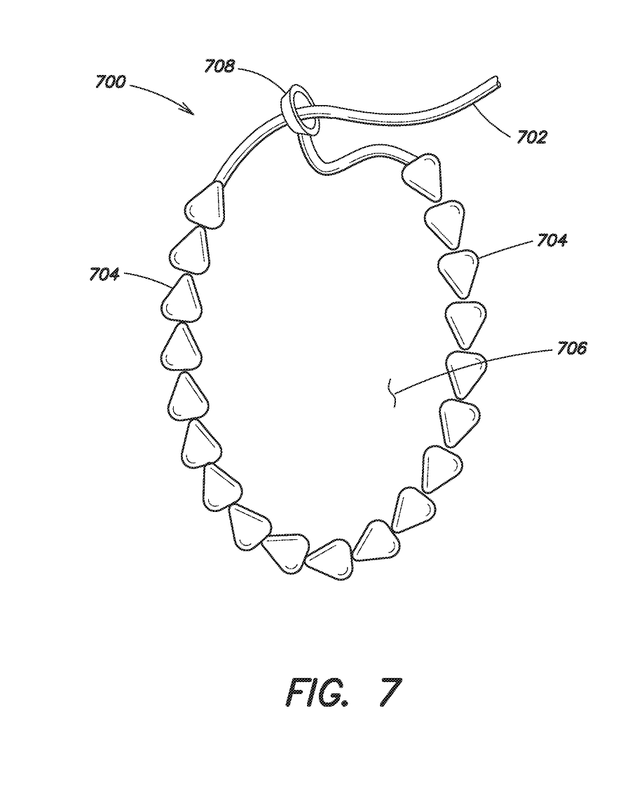

FIG. 7 is another embodiment of an LAA closure device 700. The LAA closure device 700 can include a snare wire 702 constructed and arranged of beaded sections 704 that form at least the loop portion 706 of the snare wire 702. The individual beads of the bead section are constructed and arranged to allow an engagement member 708 to slide along the snare wire shrinking the circumference of the loop portion 706, while preventing the engagement member from travelling in the opposite direction.

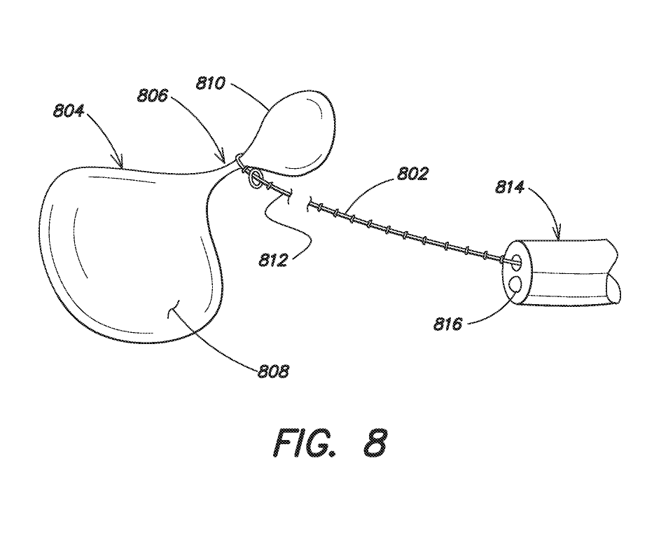

Regardless of the configuration of the snare wire, the LAA closure device can be used to perform LAA closure procedures that simplify the surgical approach, reduce the time necessary to complete the surgical procedure, and minimize the impact on the patient resulting from the surgery itself. FIG. 8 is an illustration of a snare wire 802 secured to a patient's heart 804 at the junction 806 between the LA 808 and the LAA 810. Once the snare wire has been secured at the junction 806, the circumference of the loop portion can be reduced until the LAA is completely closed off of from the LA. The distal portion 812 of the snare wire 802 can be detached, cut, or broken to leave the loop portion secured in place. The entire procedure can be visualized through one or more cameras 816 (e.g., CCDs) positioned on the end of the surgical device 814.

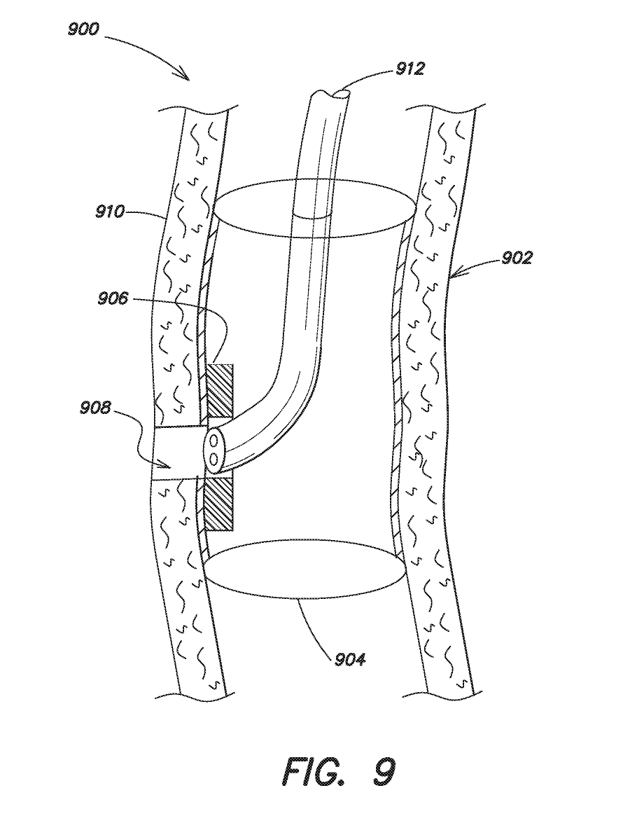

FIG. 9 is a semitransparent view of a LAA closure device 900 being advanced from a patient's esophagus 902 through a liner 904 and barrier 906 which provide a sealable opening 908 through the patient's esophageal wall 910. In some embodiments, the liner 904 can be surgically placed in a patient's esophagus to prepare a surgical field for access to the patient's thoracic cavity. The liner can be attached to the patient's esophagus to provide a sterile barrier for any surgical procedure. The barrier 906 can also be attached to the patient's esophagus and/or to the liner 904. In some examples, the barrier 906 is connected to the liner 904 and both are fixed to the patient's esophagus to prepare for the LAA closure procedure. Once the liner 904 and/or the barrier 906 are in place a surgical device 912 (e.g., a scope) can be advanced from the patient's mouth or nose into the patient's esophagus 902. In one example, upon reaching the barrier 906, the surgical device is used to cut the opening 908 through one or more of the esophageal wall 910, the liner 904, and the barrier 906. The surgical device 912 is then inserted through the barrier 906 and into the patient's thoracic cavity, where an LAA closure device can be deployed.

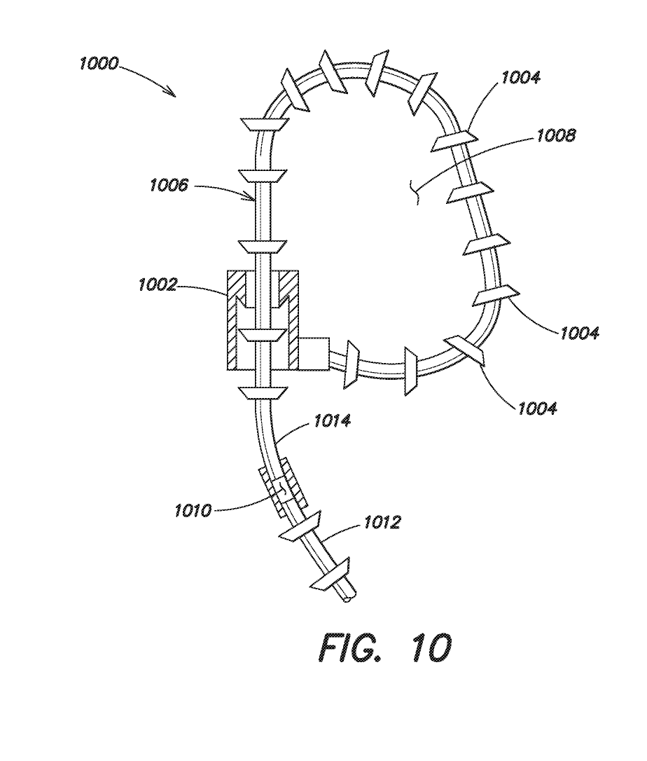

FIG. 10 is a partially transparent view of a portion of an LAA closure device 1000 according to one embodiment. In some embodiments, the engagement member of a snare wire can include a variety of structures to ensure that the loop portion of the snare wire does not slip during a procedure. In some embodiments, the closure device 1000 can include a stopper 1002 for engaging locking members 1004 disposed on a snare wire 1006. The stopper can include internal shelves that are configured to slide over the locking members 1004 in one direction while preventing movement along the snare wire 1006 in a reverse direction. In some embodiments, the locking members 1004 and the stopper 1002 are constructed and arranged such that the internal shelves engage with the locking members at the stopper as it is advanced along the snare wire to decrease the circumference of a loop portion 1008. In some embodiments, once the loop portion 1008 is reduced to a size that closes the LAA, the snare wire can be cut at a pre-configured break point 1010, separating a proximal end of the wire 1012 from the distal end 1014. Once the portions of the wire are separated, the proximal end 1012 can be withdrawn from the patient's thoracic cavity.

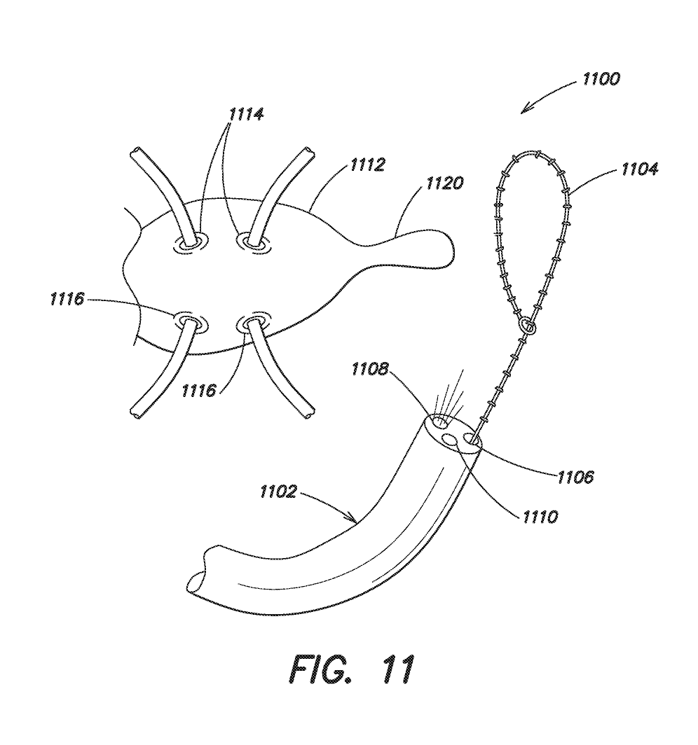

In some embodiments, LAA closure can be executed in conjunction with other procedures. In one example, LAA closure can be accompanied by extra-cardiac ablation. In some embodiments, cardiac ablation around the four pulmonary veins can be executed with the LAA closure. FIG. 11 is a view of a closure device 1100 for executing extra-cardiac ablation and LAA closure. The closure device can include a surgical instrument 1102 (e.g., scope) for carrying a snare wire 1104 or other LAA closure structure in a channel 1106 of the instrument 1102. The surgical instrument can include multiple channels and multiple surgical tools for executing procedures in the patient's thorax. For example, the surgical instrument can include a laser emitter 1108 for ablating cardiac tissue. In addition, the surgical instrument can include a CCD or camera (e.g., 1110) for visualizing a surgical field in front of the surgical instrument. Under direct visualization of the camera 1110, an operator can use the laser emitter 1108 to ablate cardiac tissue. For example, the laser 1108 can be used to ablate tissue of the LA 1112 around the four pulmonary veins shown at ablation lines 1114 and 1116. The ablation lines can be positioned to produce a greater effect around the openings of the pulmonary veins.

According to one embodiment, following ablation or during the ablation procedure, an operator can extend the snare wire 1104 about the neck of the LAA 1120 and complete a closure of the LAA from the rest of the patient's heart.

### Example Procedure

According to one embodiment, an example LAA closure or obliteration procedure can begin with insertion of a surgical instrument (e.g., a scope) into the patients' esophagus. The instrument can be advanced into the esophagus until it reaches an esophageal barrier fixed to the patient's esophagus. In some examples, the barrier includes a pre-define opening that is biased to remain closed until acted upon. The surgical instrument can be a small scope used to overcome the bias in the barrier to allow the scope to pass through the barrier and the patient's esophagus. In some embodiments, the scope carries a snare wire or other LAA closure structure in the patient's posterior mediastinum at the level of the LAA of the patient's heart.

Under visualization from cameras at the end of the surgical instrument, an operator can extend an LAA closure device (e.g., snare wire) and position the LAA closure device around the patient's LAA. In some embodiments, a loop portion of the snare wire is hooked upon the LAA and moved along the heart tissue until it is position at the connection between the LA and the LAA. The operator can then tighten the snare wire around the neck of the LAA until the physiological connections/communication between the LA and LAA are obliterated. Once LAA closure/obliteration is achieved, the loop portion of the snare wire can be disconnected. For example, the snare wire can be broken and/or cut to leave a distal portion of the wire in place around the neck of the LAA. The proximal portion of the wire and the surgical instrument can be withdrawn from the mediastinum. Upon withdrawal of the surgical instrument from the barrier, the bias to close the pre-defined opening in the barrier operates to seal the opening from the esophagus into the patient's thoracic cavity. The surgical instrument can be completely withdrawn leaving the barrier in place to protect the opening made in the patient's esophagus until complete healing takes place.

In some embodiments, the barrier can be used in conjunction with a liner for the patient's esophagus. Upon withdrawing the surgical instrument, the liner can also be removed , leaving the barrier in place to protect the incision made in the esophagus. Once healing has taken place, the barrier can be removed from the esophagus. In one alternative, the barrier can be bio-absorbed over time as the esophagus heals. In some examples, the barrier is constructed of a bio-absorbable material. The barrier can be constructed such that bio-absorption will take at least one to two days allowing the patient's esophagus to heal to the point that no contamination will occur at the entry site.

### Example Closure Device

According to some embodiments, an LAA closure device can include two rings with opposing recurved teeth connected by surgical wires disposed along the circumference of both rings. The closure device can be housed and deployed from a 2-3 mm channel in a surgical instrument (e.g., a scope) under visual guidance. In some examples, the surgical device can include cameras disposed at an end of the surgical device permitting direct visualization of the placement of the concentric rings.

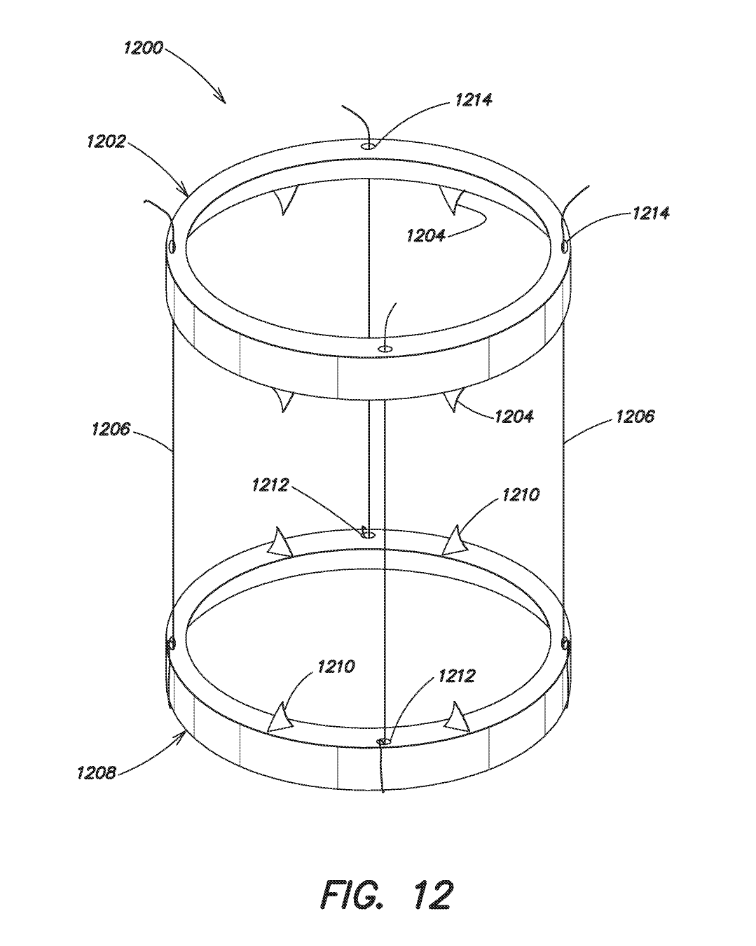

FIG. 12 is an illustration of an example LAA closure device 1200 including co-centric rings (distal ring 1202 and proximal ring 1208). Each ring includes a plurality of anchors (e.g., recurved teeth 1204 and 1210 respectively) for anchoring each ring to the tissue of the patient's heart. The anchors on the distal ring (distal from the surgical device used to deliver the rings) are configured to allow the distal ring 1202 to pass over the tissue of the LAA. Once the distal ring is positioned at the junction between the LAA and the LA, opposite motion of the ring causes the anchors (e.g., recurved teeth 1204) to seat into the heart tissue. Once the anchors are seated, the distal ring is fixed in place. The proximal ring 1210 can then be positioned at the exterior edge of the LAA. By retracting the connecting wires 1206, the wires exert pressure on the LAA, closing and/or obliteration the LAA.

In some embodiments, the wires 1206 can be threaded through guide channels 1212 in the proximal ring. The wires can be looped through connection within the distal ring 1214 such that, retraction of the wires causes the two rings to move closer together. Due to the distal rings being anchored to the neck of the LAA, the retraction of the wires 1206 can be configured to pull the proximal ring to the distal rings to close the LAA and/or obliterate the tissue of the LAA.

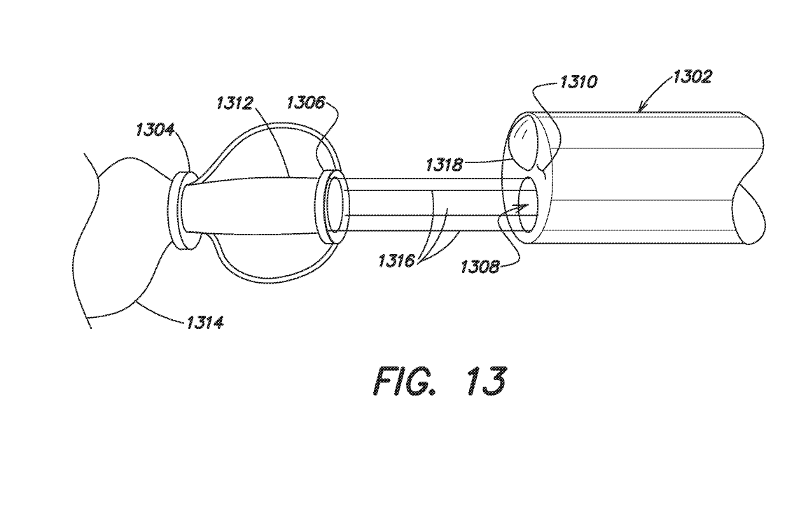

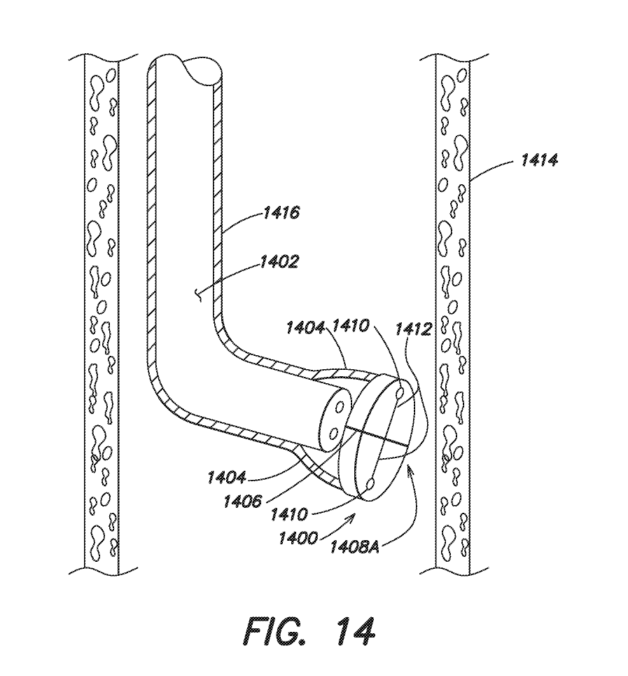

FIG. 13 is another embodiment of an LAA closure device 1300 having concentric rings. A scope 1302 or other surgical device houses the concentric rings (e.g., distal ring 1304 and proximal ring 1306). The rings can be advances out of a channel 1308 in the scope 1302. In some embodiments, the scope can include extension arms (not shown) for advancing the distal rings beyond the front surface of the scope 1310. The extension arms can be configured to push the distal ring over the LAA tissue until the distal ring 1304 is positioned at the junction between the LA 1314 and LAA 1312. In other embodiments, channel 1308 can be configured to produce a vacuum, and the LAA tissue can be drawn into channel 1308 in order to place the distal ring 1304 at the junction between the LA 1314 and the LAA 1312. The scope 1302 can be retracted leaving the distal ring anchored at the junction between the LA and LAA. The proximal ring 1306 can then be drawn towards the distal ring 1304 using, for example, tension wires 1316 to close the LAA. In some examples, the LAA is obliterated by the radial forces created by the tension wires. Proper positioning of the rings can be verified, for example, by direct visualization. In one embodiment, the scope 1302 can include a camera 1318 on its front surface 1310.

Various structures can be used to close the LAA from a posture external to the patient's heart. For example, a snare wire closure device can be housed within a surgical device (e.g., a scope) introduced into the patient's thoracic cavity. The snare wire can be used to seal the LAA tissue off from the other portions of the patient's heart. In other examples, a wire traction device can be used to close and/or obliterate the LAA portion of the patient's heart. In some examples, the wire traction device can include paired rings with anchors. The rings can be configured to generate radial pressure on the LAA to close or obliterate the LAA tissue. In some operations, the distal ring (distal to the surgical device delivering the rings to the surgical field) is able to distend to be pushed forward to end up around the neck of the LAA between the LA and LAA. Pulling on the distal ring causes anchors on the distal ring (e.g., recurved teeth) to be inserted into the tissues of the LAA, creating a stable anchor. The proximal ring can then be pushed forward until positioned at the surface of LAA. Further advancement of the proximal ring causes its respective anchors (e.g., recurved teeth) to be inserted into the tissue of LAA creating a second anchor. With both rings anchored at positions on either end of the LAA, pulling on the wires connecting and/or pushing in the proximal ring, causes radial forces created by the wires to obliterate the LAA from outside to a cylindrical structure with the same diameter of the rings (e.g., 2-4 mm) which is much smaller than the diameter of the LAA (3-4 cm). The wires can be cut between the proximal ring and the scope to allow the scope to be withdrawn.

### Embodiments for LAA Using a Barrier Device

In various embodiments, a barrier is provided that manages sterile access through a body lumen. The barrier can be deployed on the end of a surgical device (e.g., a scope). In some embodiments, the barrier is releaseably attached to the surgical device. When a desired location is reached, the barrier is attached to the body lumen (e.g., a patient's esophagus) and separated from the surgical device. Attachment can be made by suture, anchor, and/or adhesive, among other options. In one example, the barrier includes an adhesive layer that can be activated by the surgical device responsive to placement. For example, the surgical device can activate the adhesive layer chemically and/or by exposing the adhesive to light. In some embodiments, physical anchors and/or sutures can also be used in combination with the adhesive to secure the barrier to a body lumen. Once secured, pre-defined openings disposed in the barrier can be used to provide resealable access to the body lumen to generate a surgical opening and close the opening once a surgical procedure is complete.

In one example, the barrier has a pre-defined opening exposed to any lumen in which it is placed that is configured to remain closed unless acted upon. For example, a surgical device can be used to physically overcome the bias for the opening to remain closed to access the lumen to which the barrier is attached. In other examples, the barrier can include opening mechanisms that can be activated by the surgical device and/or via a control signal. Once open, the surgical device can cut through the body lumen to perform translumenal surgical procedures. In one embodiment, the barrier comprised a single contiguous layer having a pre-defined opening held closed by the bias characteristics of the material around the pre-defined opening. In another embodiment, the barrier is a unitary and uniform singular layer having a pre-defined opening.

At least some embodiments and methods include managing surgical procedures through the barrier and the lumen to which it is attached. In some embodiments, the barrier includes at least one pre-defined opening configured to remain closed unless acted upon. Further, the at least one pre-defined opening can be configured to return to the closed position and maintain the pre-defined opening in a closed position when at rest. Once released from an open position, the at least one pre-defined opening will close. Such configuration enables sterile access to a surgical field that is configured to automatically seal and protect the site of access.

Various embodiments enable procedures that can safely and accurately create a translumenal (e.g., transesophageal) access into the mediastinum, the thoracic cavity and the cervical cavity. For example, the procedures can be carried out without the need for stopping the heart, or for cardiopulmonary bypass, general anesthesia, or gross or minor thoracotomy. Translumenal access as described herein can be used to perform a variety of diagnostic and therapeutic surgical, orthopedic and neurosurgical procedures.

Shown in FIG. 1 is an example embodiment of surgical device 1402 (e.g., scope) and barrier 1400. The surgical device includes mating structures 1404 for releaseably attaching to the barrier 1400. In one example, the barrier can be constructed of rubber or other elastomeric material. According to one embodiment, the barrier 1400 includes a slit extending through the barrier 1406. The barrier can be configured to maintain the slit in a closed position 1408A until the slit is acted upon, either by the surgical device or through opening mechanisms 1140. In some examples, opening mechanisms 1140 can include paired actuators embedded in the barrier 1400. Upon activation the actuators pull internal connectors 112 (e.g., strings) toward their respective actuator. The connectors are fixed at one end to respective sides of the slit 1406, such that upon activation the actuators pull the slit 1406 open. In another example screw drive motors may be used as opening mechanisms. Once activated, the motor pulls the edges of the slit 1406 way from each other to provide an opening in the barrier 1400.

According to one embodiment, the surgical device 1402 can be used to position the barrier 1400 in a patient's esophagus 114. To preserve the sterility of the surgical device, the device 1402 can include a sterile sheath 116 that expands to maintain coverage over the surgical device as it progress into the patient's esophagus 114. The barrier 1400 can be fixed to the esophagus so as to provide a sealed opening through which transesophageal surgical procedures may be performed.

According to some embodiments, the barrier device includes a pre-defined opening extending from a first surface of the barrier device to a second surface of the barrier device. The first surface can be constructed and arranged to be uniform when the pre-defined opening is in a close position. The second surface can be attached to a body lumen. The uniform first surface improves over other approaches, for example, as the barrier can remain in place in a body lumen while limiting any impediment to flow through the body lumen. In some embodiments, the barrier is constructed of a singular layer having a uniform first surface. The singular layer and uniform surface can facilitate long term placement of the barrier device in a body lumen, while minimizing any impact on normal bodily function in the lumen.

According to one aspect, the barrier device can be configured for facilitating the controlled and reversible creation of an opening in the wall of a body lumen, for example the esophagus, through which an endoscopic device can be inserted to perform surgery in adjacent tissues, particularly in the cervical and thoracic regions. The barrier device is a structure that covers part of the lumen walls of a hollow body organ or body cavity, in proximity to an organ or a structure outside the lumen. Various embodiments and examples are described in relation to the lumen of the esophagus. However, any body lumen or cavity can be targeted with similar techniques. In one example, the barrier device allows for elongated surgical devices to be deployed through/across the lumen wall, to target extralumenal organs or structures for the performance of diagnostic and therapeutic procedures.

According to one embodiment, the barrier can be held in place on a body lumen by an adhesive and/or mechanical attachment (e.g., sutures, anchors, etc.). The barrier typically remains in place before and after a surgical procedure to cover and protect the area of the body lumen that was opened to provide surgical access to an extralumenal organ and/or space.

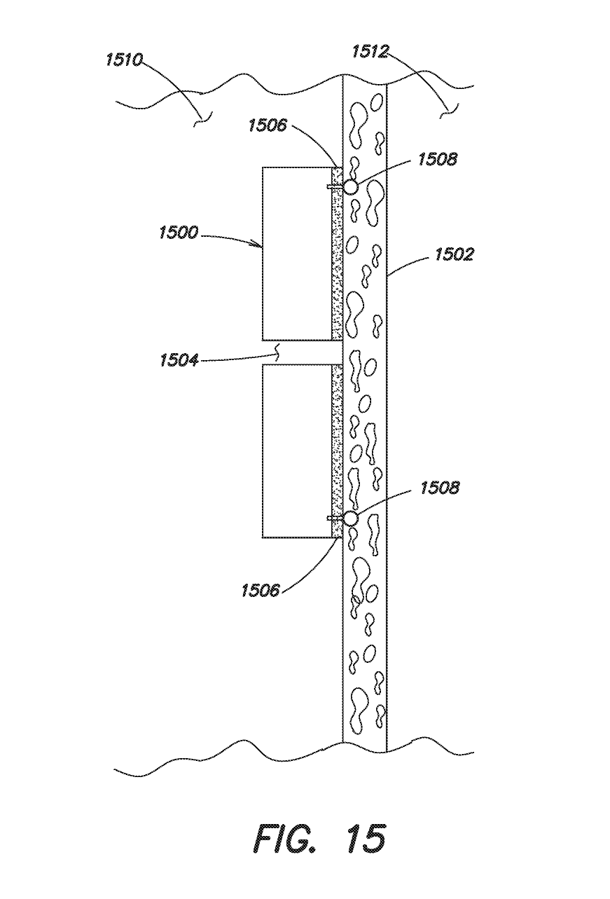

Shown in FIG. 15 is side cut out view of an example embodiment of a barrier 1500 attached to a patient's esophageal wall 1502. The barrier 1500 includes a slit 1504 or opening that extends the width of the barrier, which is configured to provide sealable access to the tissue of the esophageal wall 1502. According to one embodiment, the barrier 1500 is fixed to the esophageal wall 1502 by an adhesive layer 1506. In some embodiments, anchors 1508 can also be used to fix the barrier 1500 to the esophageal wall 1502. Once in place the barrier 1500 prevents bodily fluids or other contaminants from contacting the covered portion of the esophageal wall. In FIG. 2, slit 1504 is illustrated as partially open, however, when the barrier is in its at rest configuration the slit 1504 is closed. Once the barrier 1500 is in place, a surgical device can be inserted into the slit 1504 from the lumen of the esophagus 1510. The surgical device can be used to cut an opening through the esophageal wall 1502 to provide surgical access to the patient's extralumenal space 1512, which can include organs and/or tissue.

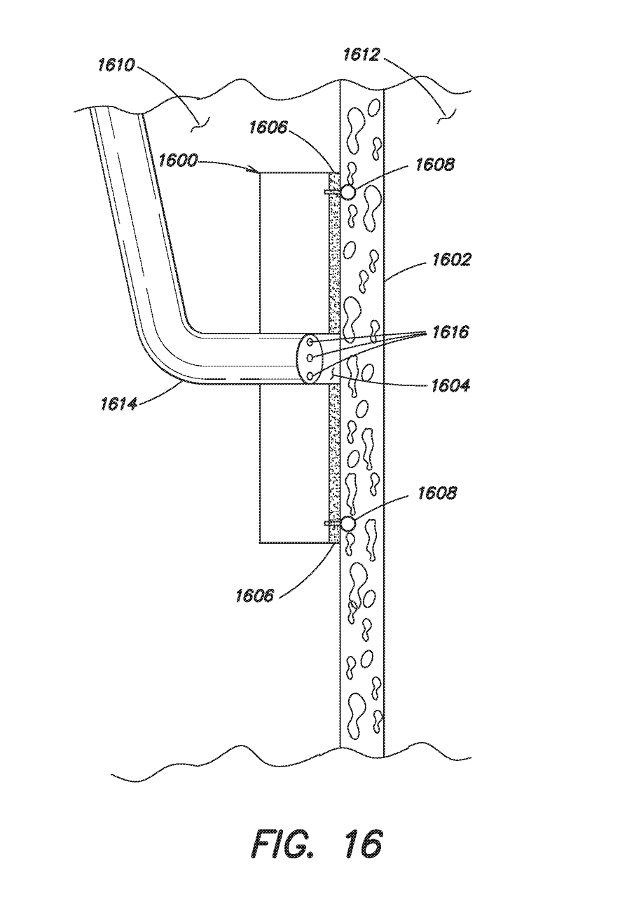

FIG. 16 shows a side cut out view of an example embodiment of a barrier 1600 attached to a patient's esophageal wall 1602. The barrier 1600 includes a slit 1604 or opening that extends the width of the barrier, which is configured to provide sealable access to the tissue of the esophageal wall 1602. In FIG. 16, a surgical device 1614 (e.g., a scope) is pushed into the opening distending the sides of opening until the barrier 1600 is in an open position with the surgical device entering at least a portion of the pre-defined opening.

According to one embodiment, the barrier 1600 is fixed to the esophageal wall 1602 by an adhesive layer 1606. In some embodiments, anchors 1608 can also be used to fix the barrier 1600 to the esophageal wall 1602. Once in place the barrier 1600 prevents bodily fluids or other contaminants from contacting the covered portion of the esophageal wall. According to one embodiment, a seal between the surgical device 1614 and the barrier is maintained as the surgical device is advanced into the opening 1604 from the lumen of the esophagus 1610. The surgical device 1614 can include a number of surgical instruments, and/or ports for delivery of chemicals, providing suction, irrigating a target area, etc. According to some embodiments, a cutting device can be extended from a port on the end of the surgical device (e.g., at 1616) to create an opening through the esophageal wall 1602 and into a space outside the esophagus 1612.

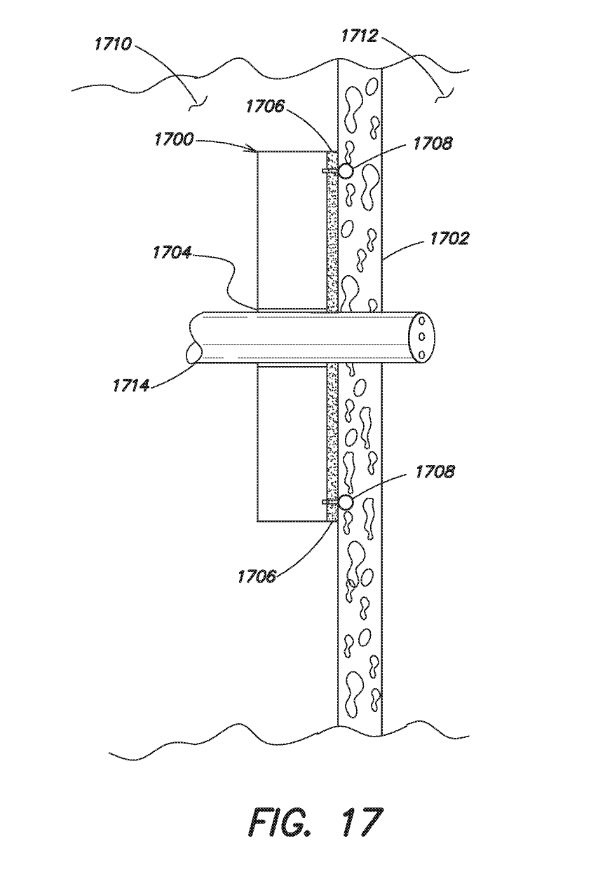

FIG. 17 shows a surgical device 1714 extended through a barrier 1700 in a cut out side view. The surgical device extends through a respective opening 1704 of the barrier 1700, and through the esophageal wall 1702. Once through the esophageal wall 1702 the surgical device can perform surgical operations within the patient's extralumenal space 1712, which can include organs and/or tissue. In one example, the barrier 1700 is fixed to the esophageal wall 1702 by an adhesive layer 1706. In some embodiments, anchors 1708 can also be used to fix the barrier 1700 to the esophageal wall 1702. Once in place, the barrier 1700 prevents bodily fluids or other contaminants from contacting the covered portion of the esophageal wall 1702 and further prevents seepage through the opening cut into the esophagus. According to one embodiment, a seal between the surgical device 1714 and the barrier is maintained as the surgical device is advanced into the opening 1704 from the lumen of the esophagus 1710 and into the extralumenal space 1712.

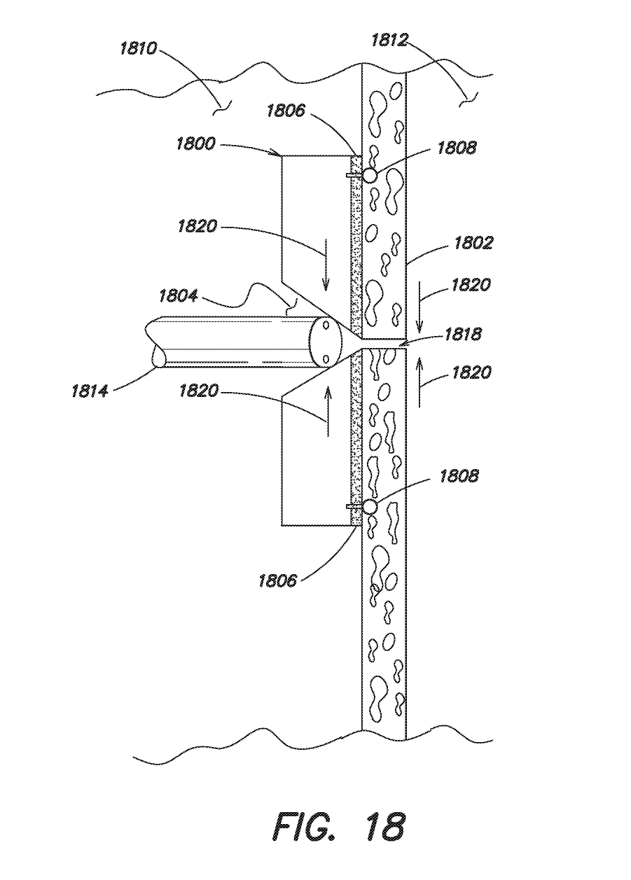

FIG. 18 shows a side cut out view of an embodiment of a barrier 1800 automatically resealing the opening in the esophageal wall 1802 and the opening 1804 in the barrier as a surgical device 1814 is withdrawn from the barrier 1800. According to some embodiments, as the surgical device 1814 is withdrawn the elastic properties of the barrier 1800 cause the edges of the opening 1804 to move together to close the opening 1804 (shown by arrows 1820). As edges of the opening 1804 are drawn together the opening 1818 in the esophageal wall 1802 is likewise closed by virtue of the barriers attached to esophageal wall 1802. An adhesive layer 1806 and/or anchors 1808 maintain a connection between the barrier 1800 and the esophageal wall such that the opening 1818 in the esophageal wall 1802 closes with the opening 1804 in the barrier 1800. According to one embodiment, utilizing a self sealing barrier 1800 enables transesophageal surgical access without contamination between the esophageal lumen 1810 and the extralumenal space 1812.

According to one aspect, minimally invasive procedures can be executed through any body lumen (e.g., the esophagus) using a barrier device. The barrier is fixed to the body lumen and used to provide a resealable opening to and through the body lumen. For example, an endoscopic instrument can be directed to a pre-defined opening in the barrier, pass through the opening, followed by passage through a portion of the body lumen to which it is attached (e.g., esophagus or other lumenal tissue). After removal of the instrument, the opening created in the body lumen can be closed automatically by the elastic properties of the barrier. The closing of the opening in the barrier likewise seals the opening made in the body lumen, and covers any incision.

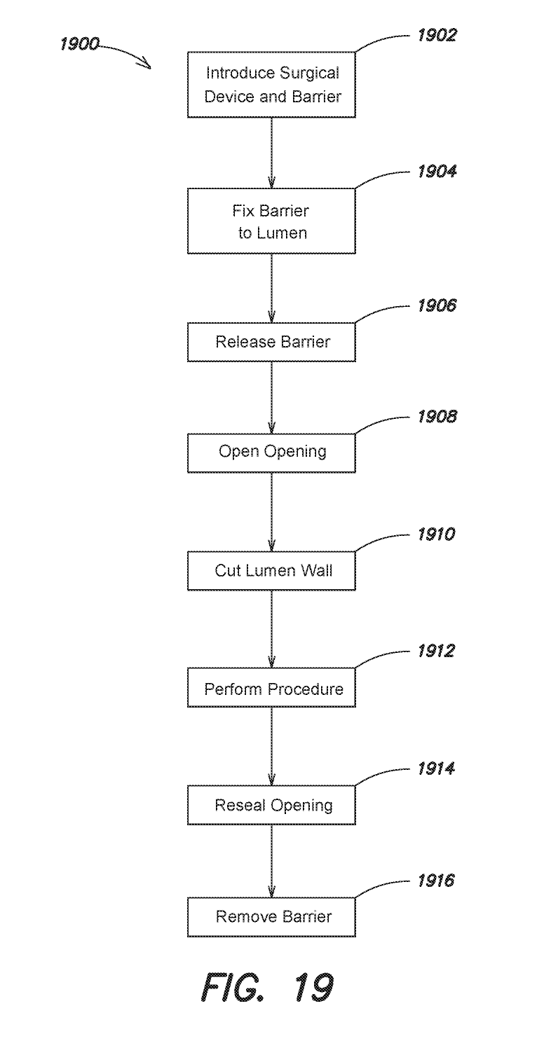

Various procedures use the barrier and a respective pre-defined opening. Shown in FIG. 19 is an example process flow 1900 for performing minimally invasive surgery through a body lumen using a barrier. Process 1900 begins at 1902 with introduction of a surgical device into a natural body opening of a patient (e.g., mouth). According to some embodiments, the barrier is attached to the surgical device at a distal end. The surgical device can be used (e.g., at 1904) to place the barrier on a desired position on the body lumen. Once correctly positioned, the barrier is fixed into position, for example, using an adhesive layer on the barrier. In some embodiments, the barrier included anchor structures to secure the barrier to the body lumen. For example, the barrier can be sutured to the lumen, welded to the lumen, stapled to the lumen, among other options.

At 1906, the barrier is released from the surgical device and at 1908 the barrier is manipulated to open a pre-defined opening in an interior portion of the barrier. In some examples, the surgical device can be advanced into the pre-defined opening. As the surgical device is advanced the barrier deforms to allow passage of the surgical device. In some examples, the barrier maintains a seal with the surgical device as it is advanced. Upon reaching the lumen wall, an incision can be made through the lumen wall (e.g., at 1910) to provide surgical access to extralumenal space.

In other embodiments, the barrier can include opening mechanisms (e.g., motors) configured to open the pre-defined opening once activated. In one embodiment, the opening mechanisms can be activated at 1908 to allow a surgical device to pass through the barrier. In some examples, motors are disposed in the barrier. The motors are connected to edges of the pre-defined opening such that the motors can open and/or close the opening. In one example, the barrier can include a switch on its surface to enable activation of the motors. A surgical device can be used to trigger the switch and open the barrier. In some embodiments, the switch can be responsive to removal of the surgical device, and close the opening as the surgical device is withdrawn from the barrier. In further examples, the surgical device can include an electrode specially configured to trigger the switch. At 1908, the electrode can be extended from the device to trigger the switch opening the opening in the barrier.

As above, once a surgical device passes through the barrier and reaches the lumen wall, an incision can be made (e.g., at 1910) and the surgical device can pass through the lumen wall and into the extralumenal space. In some embodiments, the elastic properties of the barrier can force the edges of the pre-defined opening to maintain a pressure seal against any device passing through the pre-defined opening. According to some embodiments, various surgical procedures can be performed at 1912 (e.g., cardiac ablation, cardiac mapping, valve surgery, closure of septal defects, laser surgery, delivery of energy to the heart and related structure for pacing or to enhance contractility, delivery of drugs or genetic material, and delivery of surgical devices to the heart and related structures, among other options). Once the procedure is complete the surgical device is removed from the patient. As the surgical device is being removed, the elastic properties of the barrier cause the barrier to return to a closed position at 1914. In some embodiments, the opening mechanisms can be used instead to close the opening as the surgical device is withdrawn. In further embodiments, the opening mechanism can assist the elastic properties of the barrier to close the opening as the surgical instrument is removed.

According to some embodiments, the pre-defined opening in the barrier is resealed at 1914, as well any incision made in the body lumen. The incision made in the body lumen can be drawn closed by virtue of the adhesion between the barrier and the body lumen wall. Drawing the incision in the body lumen closed facilitates healing. Further, the barrier can be left in place to protect the incision site and further promote healing. In some embodiments, the barrier is sized and constructed to be non-occlusive so it may remain in place in the body lumen for the entire period of healing of the incision. After sufficient time has passed, the barrier may be removed from the patient. In one example, process 1900 can include a removal step 1916, where the adhesive layer is exposed to any one or more of light, heat, chemical, mechanical force, or laser energy to break the bond between the barrier and the body lumen wall. In one example, the application of energy to the adhesive layer breaks down the adhesive and releases the barrier from the lumen wall.

According to some embodiments, maintaining sterility at the incision site can be a factor in a positive outcome. To facilitate a sterile surgical environment, the surgical device and/or barrier can be contained in a sterile sheath or sleeve. The sleeve can be configured to surround the surgical device and/or barrier as they are advanced into a patient and delivered to a desired location. In some examples, a distal end of the sleeve is attached to a distal end of the surgical device and/or the barrier. A proximal end of the sleeve can include a pull string that allows an operator to extend the sleeve over the surgical instrument as it is advanced into a patient. In some embodiments, the sleeve is compressed into an according shape at a distal end of the surgical devices, and the pull string is configured to deploy the sleeve over the length of the surgical device as needed.

### Example Barrier Embodiments

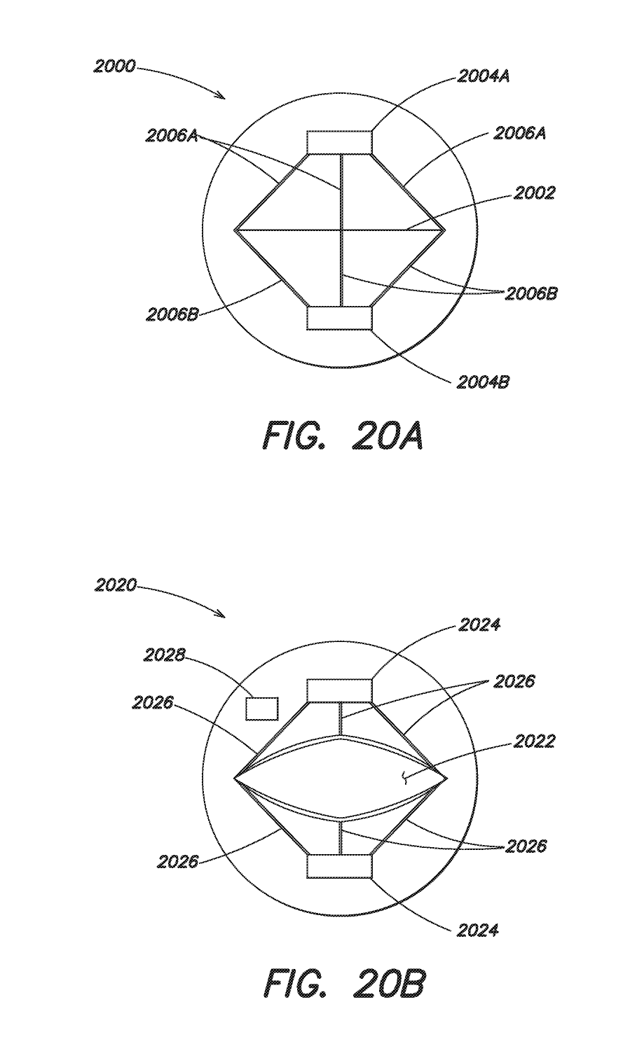

Shown in FIG. 20A is an example embodiment of a barrier 2000 having a pre-defined opening. The opening 2002 is shown in a closed position. The barrier 2000 includes actuators 2004A and 2004B which are configure to pull open the pre-defined opening 2002 through connectors 2006A and 2006B respectively. The actuators 2004A-B can be configured to operate in parallel, such that activation causes both actuators to pull on respective edges of the pre-defined opening 2002. The actuators can be motors that operate on the connectors to open the barrier 2000. In some examples, the motors can operate on strings or fibers connected to the edges of the pre-defined opening, to pull open the barrier. In other embodiments, the motors can be connected to drive shafts that operate to open and close the barrier. In further embodiments, the activations can operate on portions of the respective edges of the pre-defined opening and pull on the portions in sequence to partially open the predetermined opening as a surgical device is advanced into the opening.

FIG. 20B illustrates an example embodiment of a barrier 2020 having a pre-defined opening 2022 in an open position. The barrier 2020 includes actuators 2024 which have acted upon opening 2022 to transition the barrier into an open position via connectors 2026. In some embodiments, the barrier can include a sensor 2028 to detect the presence of a surgical device at the opening 2024. In example, the sensor is configured to detect deformation of the barrier 2020 responsive to the surgical device being advanced into the opening 2022. Once the deformation is detected by the sensor, the sensor triggers the actuators to assist in opening the barrier at 2022. In other embodiments, the sensor can be configured to detect physical contact with a surgical device and trigger the actuators responsive to physical contact.

### Example Surgical Fields

According to various embodiments, the barrier device can be inserted through a natural body opening of a patient (e.g., mouth, anus, vagina, ear, nose, etc.) to access body lumens. The barrier device can be placed within respective body lumens to manage sterile translumenal access for medical procedures. In some embodiments, the barrier device to can be used to manage translumenal access enabling procedures on the patient's heart and/or other organs or tissues in the intra-thoracic cavity using one or more devices introduced through the pre-defined opening in the barrier device and through the lumen to which it is attached.

The surgical procedures can include, for example, manipulation and/or surgical intervention of the patient's lungs, pleura, pulmonary vessels, bronchi, trachea and related structures. In other embodiments, the surgical procedures can include manipulation and/or surgical intervention on the patient's great vessels of the thorax, as well as, manipulation and/or surgical intervention on the patient's lymph nodes and lymphatic vessels of the thorax.

Further embodiments, can include surgical procedures for manipulation and/or surgical intervention on the patient's nerve roots and nerves of the thorax, manipulation and/or surgical intervention on the patient's ribs, intercostal tissues and structures, thoracic cartilaginous structures and muscles of the thoracic wall, manipulation and/or surgical intervention on the patient's thoracic and cervical vertebrae, intervertebral disks, foramina, nerve roots, paravertebral muscles, vertebral vessels and structures related to cervical and thoracic vertebrae.

In still other embodiments, the barrier and surgical devices can be configured for manipulation and/or surgical intervention on the patient's mediastinum and related mediastinal structures, or manipulation and/or surgical intervention on the patient's esophageal wall and paraesophageal structures. In other embodiments, other surgical procedures can be performed using the barrier device and associated surgical devices.

According to some embodiments, a barrier enables minimally invasive surgical procedures. The barrier device can be removeably attached to a distal end of a surgical device to facilitate placement, and the execution of the surgical procedure. In one example, the barrier is positioned so that the pre-defined opening faces anteriorly to the heart, and is used with surgical devices for a variety of procedures including atrial mapping and ablation, treatment of arrhythmia, valvular heart disease treatments, occlusion of septal defects, LAA, etc. In other examples, the pre-defined opening may be facing anteriorly towards the lungs and anterior mediastinum for a variety of procedures both diagnostic or therapeutic, directly or in related structures, with the procedures including biopsy, tumor staging, imaging, injection, delivery of materials, cryotherapy, RF treatment, and laser treatment on tissues including lungs, great vessels, trachea, LN, esophagus, nerves, diaphragm, and lymphatics.

According to some embodiments, a surgical device coupled to the barrier can include imaging structures. The imaging structure can be used to facilitate placement of the barrier in a body lumen. In some examples, the barrier is constructed of a translucent material that enables imaging through the barrier. In further examples, an imaging apparatus can be extended into the barrier at a respective opening, such that the imaging apparatus extends through the opening to provide visual information on placement of a barrier (e.g., regardless of whether the barrier is translucent).

In further embodiments, the barrier can include processing capability and/or be connected to computer system that provides processing capability. In some examples, computer systems can be used to control opening and closing of pre-defined openings in barrier devices. For example, deformation sensors embedded in a barrier can deliver sensor information to a processor configured to analyze the sensor information to determine proximity of a surgical device. Responsive to determining that a surgical device is present at the pre-defined opening, the processor can be configured to open and/or close the pre-defined opening. In other embodiments, electrodes on the surgical device can be used to contact the barrier. Responsive to detecting contact at the barrier, the processor can be configured to trigger opening of the pre-defined opening. The absence of the electrodes can likewise be determined by sensor information received by the processor. Once the absence is determined the processor can be configured to trigger the pre-defined opening to close. In further embodiments, the actions triggered by the processor (e.g., open and/or close) can be responsive to positioning information of a surgical instrument within a pre-defined opening of a barrier. In some examples, sensors within the barrier can provide information on a penetration depth of a surgical device and control associated actuators accordingly.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. An apparatus for executing a left atrial appendage operation, the apparatus comprising:
a surgical instrument constructed and arranged to pass through a sealable opening at an esophageal barrier, including a closure component configured to attach to an exterior portion of a patient's heart, and to collapse a portion of the heart at the exterior portion of the patient's heart to close the left atrial appendage (“LAA”).

2. The apparatus of claim 1, wherein the closure component comprises a snare wire configured to extend about the portion of the patient's heart.

3. The apparatus of claim 2, wherein the snare wire comprises a wire having a loop portion, a locking portion, and a plurality of teeth.

4. The apparatus of claim 3, wherein the plurality of teeth are constructed and arranged to directionally engage the locking portion.

5. The apparatus of claim 4, wherein the plurality of teeth are constructed and arranged to pass through the locking potion in one direction and prevent passage through the locking portion in an opposite direction.

6. The apparatus of claim 4, wherein the locking portion is configured to engage subsequent ones of the plurality of teeth to reduce the loop portion in size responsive to retraction of the snare wire.

7. The apparatus of claim 6, wherein the plurality of teeth are constructed and arranged such that the loop portion is reduced to a diameter of less than 2 mm.

8. The apparatus of claim 1, wherein the closure component comprises at least a first ring and a second ring.

9. The apparatus of claim 8, wherein the at least a first ring and a second ring comprise at least a distal ring and a proximal ring, wherein the distal ring is configured to distend, such that the distal ring is configured to be advanced over tissue of the LAA, such that a portion of the tissue of the LAA pass through the distal ring.

10. The apparatus of claim 9, wherein the distal ring comprises a plurality of unidirectional anchors.

11. The apparatus of claim 10, wherein the plurality of unidirectional anchors are constructed and arranged such that the plurality of unidirectional anchors are oriented to engage the tissue of the LAA and anchor the distal ring against a movement of the distal ring in an opposite direction of the advancement of the distal ring over the tissue of the LAA.

12. The apparatus of claim 8, wherein the proximal ring includes a plurality of anchors configured to engage tissue of the LAA.

13. The apparatus of claim 8, further comprising connection wires between the distal and the proximal rings.

14. The apparatus of claim 13, wherein the connection wires are configured to apply radial pressure to the tissue of the LAA to close the LAA.

15. A method for a left atrial appendage operation, the method comprising:
introducing a surgical device through a sealable opening in an esophageal barrier into a cardiac cavity;
advancing a closure component of the surgical device into a thoracic cavity of a patient's heart;
capturing, from the thoracic cavity, a portion of a patient's heart including a left atrial appendage with the closure component; and
collapsing the portion of the patient's heart using the closure component.

16. The method according to claim 15, wherein capturing, from the thoracic cavity, the portion of a patient's heart with the closure component includes extending a snare wire about the portion of the patient's heart.

17. The method according to claim 16, further comprising retracting the snare wire, thereby constricting the portion of the heart of the patient.

18. The method according to claim 15, wherein capturing, from the thoracic cavity, the portion of a patient's heart with the closure component includes extending a distal ring over the tissue of the LAA.

19. The method according to claim 18, further comprising anchoring the distal ring at the junction between the LAA and the left atrium (“LA”).

20. The method according to claim 19, further comprising anchoring a proximal ring on a surface of the LAA.

21. The method according to claim 20, wherein collapsing the portion of the patient's heart using the closure component includes exerting radial pressure on the tissue of the LAA with wires connecting the distal and proximal rings.
